(19) United States
(12) Patent Application Publication
(10) Pub. No.: US 2025/0000397 A1
(43) Pub. Date: Jan. 2, 2025

(54) LOW NOISE OXIMETRY CABLE

(71) Applicant: Masimo Corporation, Irvine, CA (US)

(72) Inventors: Benjamin C. Triman, Rancho Santa Margarita, CA (US); John C. Schmidt, Lake Forest, CA (US)

(21) Appl. No.: 18/765,634

(22) Filed: Jul. 8, 2024

Related U.S. Application Data
(63) Continuation of application No. 17/807,193, filed on Jun. 16, 2022, now Pat. No. 12,053,280.
Application No. 17/807,193 is a continuation of application No. 16/598,385, filed on Oct. 10, 2019, now Pat. No. 11,389,093.
(60) Provisional application No. 62/744,308, filed on Oct. 11, 2018.

(51) Int. Cl. A61B 5/1455; H01B 7/04; H01B 7/18

(52) U.S. Cl. CPC A61B 5/14551; H01B 7/04; H01B 7/1805; H01B 7/1875; A61B 2562/12; A61B 2562/222

(57) ABSTRACT

The present disclosure includes a cable for a patient monitoring system. The cable can have a flexible and durable overall construction that enables the cable to withstand repeated winding and unwinding and prevent kinks from developing. The cable may include multiple bundles encased in inner jackets that reduce the amount of friction against other cable elements and allows the bundle to move more freely inside an outer jacket of the cable. The multiple bundles may include multiple wires or cords. The cable can include a flexible core that runs through the middle of the cable. The multiple bundles can be twisted, weaved, or untwisted around the core.

### RELATED APPLICATIONS

Any and all applications for which a domestic priority claim is identified in the Application Data Sheet of the present application are hereby incorporated by reference under 37 CFR 1.57.

### BACKGROUND

### Field of the Disclosure

The disclosure relates to improving the performance of patient monitors through low noise cabling.

### Description of the Related Art

Oximetry utilizes a noninvasive optical sensor to measure physiological parameters of a patient. In general, the sensor has light emitting diodes (LEDs) that transmit optical radiation into a tissue site and a detector that responds to the intensity of the optical radiation after absorption (for example, by transmission or transreflectance) by, for example, pulsatile arterial blood flowing within the tissue site. Based on this response, a processor determines measurements for oxygen saturation (SpO2), pulse rate, plethysmograph waveforms, perfusion quality index (for example, an index that quantifies perfusion), assessments of other blood constituents, parameters or analytes, including for example, a percent value for arterial carbon monoxide saturation (HbCO), a percent value for methemoglobin saturation (a brownish-red form of hemoglobin that cannot function as an oxygen carrier) (HbMet), total hemoglobin (HbT), fractional SpO2 (SpaO2) or the like.

Additionally, caregivers often desire knowledge of HbO₂, Hb, blood glucose (HbGu), water, the presence or absence of therapeutic drugs (aspirin, Dapson, nitrates, or the like) or abusive/recreational drugs (methamphetamine, alcohol, steroids, or the like), concentrations of carbon dioxide (CO₂), oxygen (O₂), oxygen concentration, pH levels, bilirubin, perfusion quality, albumin, cyanmethemoglobin, and sulfhemoglobin (HbSulf), signal quality or the like.

It is noted that “oximetry” as used herein encompasses its broad ordinary meaning known to one of skill in the art, which includes at least those noninvasive procedures for measuring parameters of circulating blood through spectroscopy. Moreover, “plethysmograph” as used herein (commonly referred to as “photoplethysmograph”), encompasses its broad ordinary meaning known to one of skill in the art, which includes at least data representative of a change in the absorption of particular wavelengths of light as a function of the changes in body tissue resulting from pulsing blood.

Oximeters capable of reading many of the foregoing parameters during motion induced noise are available from Masimo Corporation of Irvine, California. Moreover, portable and other oximeters are disclosed in at least U.S. Pat. Nos. 6,770,028, 6,658,276, 6,157,850, 6,002,952, and 5,769,785, which are incorporated by reference herein, and others patent publications such as those listed at http://www.masimo.com/patents.htm. Such reading through motion oximeters have gained rapid acceptance in a wide variety of medical applications, including surgical wards, intensive care and neonatal units, general wards, home care, physical training, and virtually all types of monitoring scenarios.

The detectors of the noninvasive sensors read by many of the foregoing patient monitors generate one or more low-level signals that are susceptible to corruption from various noise, such as electromagnetic interference (EMI) and internal noise that originate in the sensor, cabling and monitors. One internal noise source is due to a triboelectric effect, which includes static charges that build when two materials rub together. For example, when a cable housing detector wires is flexed, impacted, or the like, the detector wires may rub together and triboelectric noise can be induced in the detector signal. These induced triboelectric noise spikes can be orders of magnitude larger than the desired low level detector signals.

To alleviate the buildup of triboelectric charges, low noise cable manufacturers included graphite coatings exterior to, for example, the cabling configured to communicate detector signals. However, the graphite gel used in the manufacturing process proved difficult to apply and remove. Because of these and other difficulties, manufacturers began substituting the graphite coatings with a coextruded conductive PVC sheath around, for example, their sensitive signal carrying cables.

### SUMMARY

The present disclosure describes a low noise oximetry cable that provides an improved, flexible, and durable overall construction for various medical environments. The cable can be particularly useful in environments such as emergency medical situations (EMS), where cables can undergo a tremendous amount of stress, such as repeated winding and unwinding, that can cause kinks to develop leading to or creating an electrical short in the cables. The construction of the cable described herein can reduce damage resulting from stress like repeated winding and unwinding and prevent kinks from developing. The cable can be part of a patient monitoring system that transmits real-time patient data to individual or multiple integrated or non-integrated devices.

The cable can include multiple bundles of conductors. The bundles can evenly distribute stress exerted on the conductors or cords within the cable. The bundles may be wrapped or weaved around a core that is flexible and durable. Some or all of conductors may be assembled twisted or untwisted with one or more cords. The conductors may be assembled twisted or untwisted around one or more central cores or cords.

An oximetry system is disclosed that can acquire signals indicative of one or more physiological parameters of a patient. The oximetry system can include a noninvasive sensor including a detector configured to detect light attenuated by a body tissue and output a detector signal indicative of the light detected after attenuation by the body tissue. The oximetry system also includes a patient monitor configured to receive the detector signal and determine one or more physiological parameters the patient from the detector signal. The oximetry system additionally includes a cable having an outer jacket, an outer shield, a first bundle including wires, a second bundle including wires, and a core at least partially surrounded by the first bundle and the second bundle. The outer shield at least partially surrounds the first bundle and the second bundle, and the outer jacket at least partially surrounds the outer shield.

A cable is disclosed that can transmit signals for an oximetry system usable to determine of one or more physiological parameters of a patient. The cable can include a first bundle with wires. The cable also can include a second bundle with wires. The cable can additionally include a core at least partially surrounded by the first bundle and the second bundle.

In some embodiments, an oximetry system is disclosed that can acquire signals indicative of one or more physiological parameters of a patient. The oximetry system can include a noninvasive sensor, a patient monitor, and a cable. The noninvasive sensor can include a detector configured to detect light attenuated by a body tissue of a patient and output a detector signal indicative of the light detected after attenuation by the body tissue. The patient monitor can be configured to receive the detector signal and determine one or more physiological parameters for the patient from the detector signal. The cable can include an outer jacket, an outer shield, a first bundle, and a second bundle. The first bundle can include a first plurality of wires. The second bundle can include a second plurality of wires. The outer shield can at least partially surround the first bundle and the second bundle. The outer jacket can at least partially surround the outer shield.

The oximetry system of the preceding paragraph can include one or more of the following features: The noninvasive sensor can include a sensor housing configured to position an emitter and the detector proximate to the body tissue. The one or more physiological parameters can include an oxygen saturation and a pulse rate. The cable can include a core configured to reduce a tensile stress exerted on the cable and increase a durability of the cable. The core can be flexible. The first bundle and the second bundle can be twisted or weaved around the core. The first bundle can include a first shield at least partially surrounding the first plurality of wires. The cable can include a third bundle at least partially surrounded by the outer shield. The third bundle can include a third plurality of wires. The first bundle, the second bundle, and the third bundle can be twisted or weaved around a core. The first bundle and the second bundle may not be concentric with each other. The cable can include a core, and the first bundle and the second bundle may not be concentric with the core. The first bundle can include a different number of wires than the second bundle. The core can include a plurality of arcuate surfaces that contact the first bundle and the second bundle and limit an amount of contact between the first bundle and the second bundle. The plurality of arcuate surfaces can be formed at least partly by pressure from the first bundle and the second bundle on the core.

A method of manufacturing the oximetry system of preceding two paragraphs is additionally disclosed.

In some embodiments, a cable is disclosed that can transmit signals in an oximetry system. The cable can include a first bundle, a second bundle, and a core. The first bundle can include a first plurality of wires. The second bundle can include a second plurality of wires. The core can be configured to reduce a tensile stress exerted on the cable and increase a durability of the cable.

The cable of the preceding paragraph can include one or more of the following features: The first bundle and the second bundle can be twisted or weaved around the core. The first bundle can include a first shield at least partially surrounding the first plurality of wires. The second bundle can include a second shield at least partially surrounding the second plurality of wires. A third bundle that can be disposed within a jacket along with the first bundle and the second bundle. The first bundle and the second bundle may not be concentric with each other. The first bundle and the second bundle may not be concentric with the core. The core can define a central axis. The first bundle can include a different number of wires than the second bundle.

A method of manufacturing the cable of preceding two paragraphs is additionally disclosed.

In some embodiments, a method is disclosed for manufacturing a cable that can transmit signals in an oximetry system. The method can include: assembling a first bundle that can include a first plurality of wires; assembling a second bundle that can include a second plurality of wires; and placing, within a jacket, the first bundle and the second bundle adjacent to a core.

For purposes of summarizing the disclosure, certain aspects, advantages and novel features are discussed herein. It is to be understood that not necessarily all such aspects, advantages or features will be embodied in any particular embodiment of the invention and an artisan would recognize from the disclosure herein a myriad of combinations of such aspects, advantages or features.

### BRIEF DESCRIPTION OF THE DRAWINGS

FIG. 1 is a block diagram of a patient monitoring system including a patient monitor and a noninvasive optical sensor communicating through a cable.

FIG. 2A is a cutaway side-view of the cable of FIG. 1.

FIG. 2B is a cross-sectional view of the cable of FIG. 1.

FIG. 2C is a cutaway perspective view of the cable of FIG. 1.

### DETAILED DESCRIPTION

### Introduction

A low noise oximetry cable is provided that can communicate a low level sensitive signals between a sensor and a patient monitor. Wires or cords of the cable can be twisted within individual bundles. The individual bundles can be encased within an inner jacket that can reduce friction between different cable elements. The bundles can be encased at least partially by an inner shield that may reduce EMI between the bundles and crosstalk with other cables.

The bundles can be disposed within an outer jacket and an outer shield. The bundles can wrap around a core. The core can be placed in the center of the cable and surrounded by the bundles so that the core can absorb stress exerted on the cable and various cable elements. The core can be composed of a flexible or durable material. Two, three, four, or more bundles can wrap around the core.

A patient monitor usable with the cable disclosed herein is the Root® Platform, a patient monitoring and connectivity platform available from Masimo Corporation, Irvine, CA. A mobile physiological parameter monitoring system usable with the cable is described in U.S. Pat. No. 9,436,645, issued on Sep. 6, 2016, titled “MEDICAL MONITORING HUB,” the disclosure of which is hereby incorporated by reference in its entirety.

To facilitate a complete understanding of the disclosure, the remainder of the detailed description references the drawings, wherein like references number are references with numerals throughout.

### Patient Monitoring System Environment

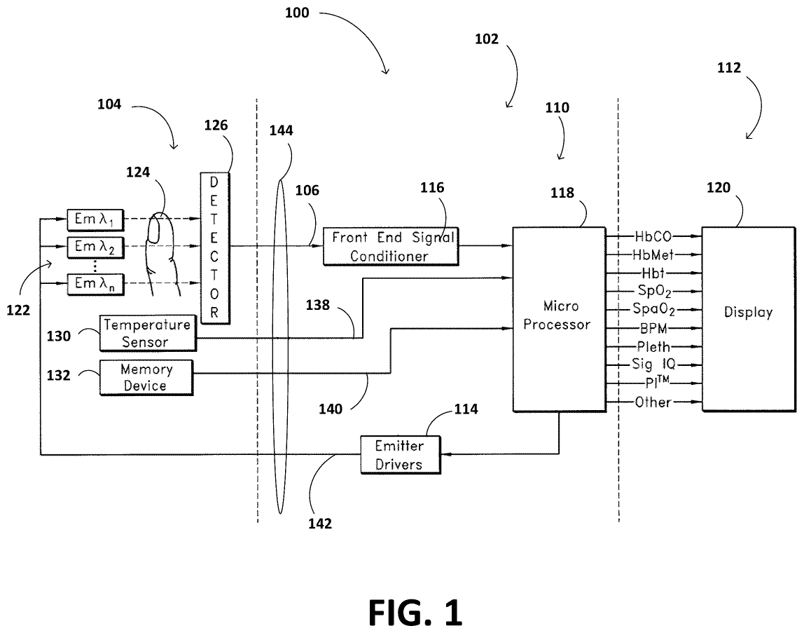

FIG. 1 illustrates a block diagram of a patient monitoring system 100 including a patient monitor 102 and a noninvasive optical sensor 104 communicating through a cable 144. The patient monitor 102 can include one or more processing boards 110 communicating with a host instrument 112. The one or more processing boards 110 can include processing circuitry arranged on one or more printed circuit boards capable of installation into a handheld or other monitor, or capable of being distributed as an OEM component for a wide variety of host instruments monitoring a wide variety of patient information. As shown, the one or more processing boards 110 can include an emitter driving circuit 114, a front end 116, and a microprocessor 118.

The emitter driving circuit 114 can output drive signals to the noninvasive optical sensor 104. The emitter driving circuit 114 may drive two (2) or more emitters capable of emitting light at two (2) or more wavelengths, or it may drive a matrix of eight (8) to sixteen (16) or more emitters capable of emitting light at eight (8) to sixteen (16) or more wavelengths.

The front end 116 can condition the signals, applies gain, converts signals to digital information, and the like, although any or all of the functions of the emitter driving circuit 114 and the front end 116 could be performed by other software or hardware components, or by the microprocessor 118. The microprocessor 118 may include one or more hardware or software components capable of executing instructions designed to control drive signals and to process incoming signal data related to the drive signals to determine desired physiological parameters of a monitored patient. Such parameters may include one or more of SpO₂, perfusion quality index, pulse rate, HbCO, HbMet, HbT, SpaO₂, HbO₂, Hb, HbGu, water, the presence or absence of therapeutic drugs or abusive/recreational drugs, CO₂, O₂, pH levels, bilirubin, albumin, cyanmethemoglobin, and HbSulf, signal quality, signal confidence measures, trend data on one, some, all, or combinations of the foregoing, or the like. Moreover, the microprocessor 118 can determine when alarm conditions exist for alerting a caregiver to the current condition of the patient.

The host instrument 112 can include a display device 120 capable of providing indicia representative of the calculated physiological parameters. The host instrument 112 can include virtually any housing, including a handheld or otherwise portable monitor capable of conveying one or more of the foregoing measured or calculated parameters to a caregiver. The host instrument 112 may include audio or visual alarms that alert caregivers that one or more physiological parameters are falling below or above predetermined safe thresholds, or are trending in a predetermined direction (good or bad). The host instrument 112 may include indications of the confidence a caregiver should have in the conveyed data.

The noninvasive optical sensor 104 can include emitters 122 irradiating a body tissue 124 with light, and one or more detectors 126 capable of detecting the light after attenuation by the body tissue 124. The noninvasive optical sensor 104 can also include a temperature sensor 130, such as, for example, a thermistor or the like, and a memory device 132. The memory device 132 may include any one or more of a wide variety of memory devices, including an EPROM, an EEPROM, a flash memory, a ROM, a RAM, single wire memories, combinations, or the like. The memory device 132 can store some or all of a wide variety data and information, including, for example, information on the type or operation of the sensor, type of patient or body tissue, buyer or manufacturer information, sensor characteristics including the number of wavelengths capable of being emitted, number of emitters, emitter specifications, emitter operational characteristics, emitter drive requirements, history of the sensor temperature, current, or voltage, demodulation data, calculation mode data, calibration data, software such as scripts, executable code, or the like, sensor electronic elements, sensor life data indicating whether some or all sensor components have expired and should be replaced, encryption information, keys, indexes to keys, the parameters the sensor is intended to measure (for example, HbCO, HbMet, etc.), monitor or algorithm upgrade instructions or data, some or all of parameter equations, combinations of the same, or the like.

As shown in FIG. 1, the cable 144 can communicate signals between the noninvasive optical sensor 104 and the one or more processing boards 110. The cable 144 can include one or more conductors including detector composite signal conductor(s) 106, temperature sensor conductor(s) 138, memory device conductor(s) 140, emitter drive signal conductor(s) 142, and the like. An example of the cable 144 is disclosed in U.S. Pat. No. 7,919,713, which is incorporated by reference herein.

Low-Noise Cable with Bundles

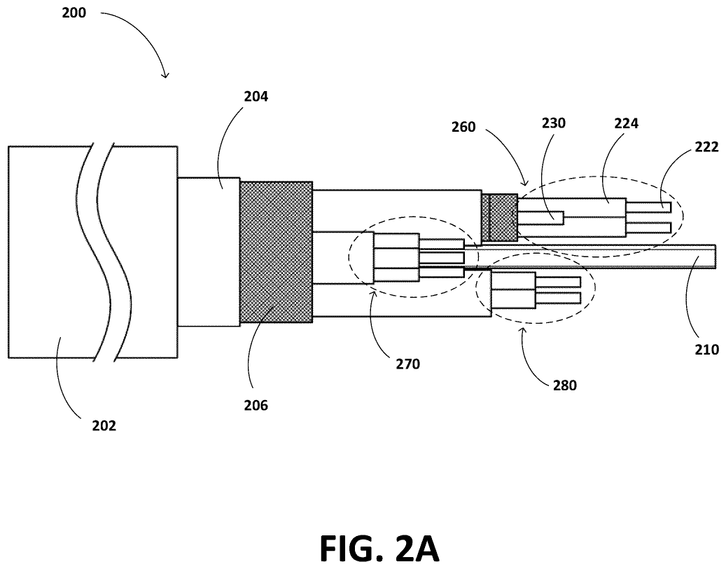

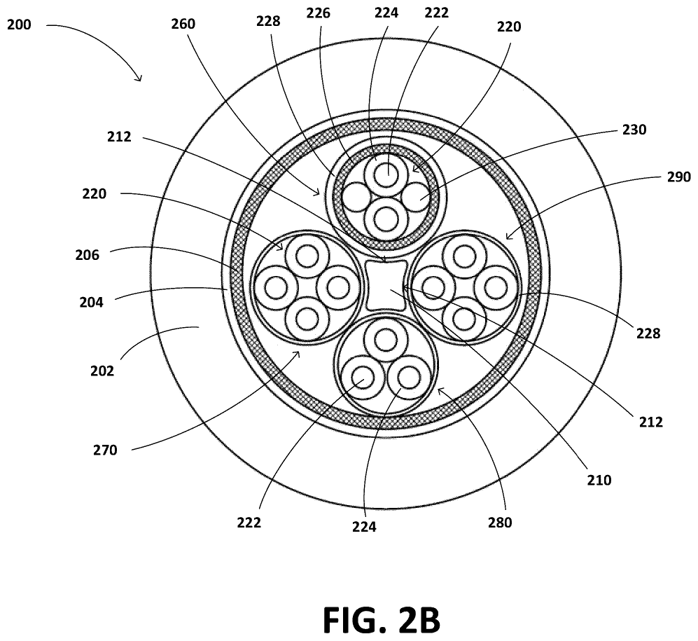

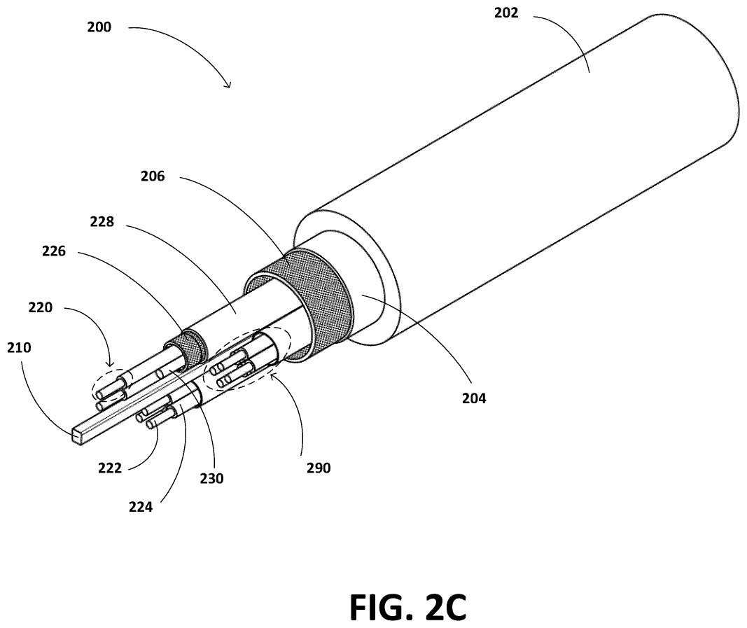

With reference to FIGS. 2A-2C, a patient cable 200 is disclosed. The cable 200 can be an example of the cable 144 of FIG. 1. The cable 200 can include an outer jacket 202, a separator 204, an outer shield 206, a first bundle 260, a second bundle 270, a third bundle 280, and a fourth bundle 290. The bundles 260, 270, 280, 290 can each include cords 230 or wires 220. The bundles 260, 270, 280, 290 can each include one or more cords 230 and one or more wires 220. The wires 220 can each include a conductor 222 encased by an insulator 224. The cable 200 can include a core 210 that is located adjacent to or between the bundles 260, 270, 280, 290.

The number of the bundles in the cable 200 can vary. The cable 200 can, for example, include two or three bundles instead of four. The number of the bundles in the cable 200 can vary depending upon desired cable thickness, shape, size (such as of an outer diameter), rigidity or flexibility, conductive performance, cost, number of signals transmitted, or the like.

The bundles 260, 270, 280, 290 can each include one or more of the wires 220. The number of the wires 220 in one of the bundles 260, 270, 280, 290 can vary depending upon desired bundle thickness, shape, size (such as outer diameter), rigidity or flexibility, conductive performance, cost, or the like. To give individual of the bundles 260, 270, 280, 290 a substantially circular shape while maintaining or improving triboelectric drain and substantially improving flexibility, the bundles 260, 270, 280, 290 can each include three or four of the wires 220 as shown in FIG. 2B. The bundles 260, 270, 280, 290 can alternatively each include two of the cords 230 and two of the wires 220. The bundles 260, 270, 280, 290 can alternatively each include five or more of the wires 220 or five or more of the cords 230. The total number of the wires 220 and the cords 230 in a given bundle can be more than four, five, six, seven, eight, nine, or ten.

The first bundle 260 can include two of the wires 220 and two of the cords 230, while the second bundle 270 and the fourth bundle 290 may each include four wires 220 but no cords 230. Moreover, the third bundle 280 may include three wires 220 and no cords 230. The total number of wires 220 or cords 230 can be the same or vary between one or more of the bundles 260, 270, 280, 290.

The cords 230 can each have an outer diameter ranging between about 0.015 inches and about 0.03 inches, between about 0.018 inches and about 0.028 inches, between about 0.02 inches and about 0.026 inches, between about 0.022 inches and about 0.024 inches, or about 0.015 inches, about 0.018 inches, about 0.02 inches, about 0.022 inches, about 0.024 inches, about 0.026 inches, about 0.028 inches, about 0.03 inches, or ranges between any two of aforementioned values. The outer diameter of each of the cords 230 can be less the about 0.015 inches or greater than about 0.03 inches. The cords 230 can each have an outer diameter of about 0.015 inches.

The wires 220 can each have an outer diameter ranging between about 0.02 inches and about 0.04 inches, between about 0.022 inches and about 0.038 inches, between about 0.024 inches and about 0.036 inches, between about 0.026 inches and about 0.034 inches, between about 0.028 inches and about 0.032 inches, or about 0.02 inches, about 0.022 inches, about 0.024 inches, about 0.026 inches, about 0.028 inches, about 0.03 inches, about 0.032 inches, about 0.034 inches, about 0.036 inches, about 0.038 inches, about 0.04 inches, or ranges between any two of aforementioned values. The outer diameter of each of the wires 220 can be less than about 0.02 inches or greater than about 0.04 inches. The wires 220 can each have an outer diameter of about 0.022 inches.

The bundles 260, 270, 280, 290 can each have a circumferential width ranging between about 0.04 inches and about 0.07 inches, about 0.045 inches and about 0.065 inches, about 0.05 inches and about 0.06 inches, about 0.054 inches and about 0.056 inches, or about 0.04 inches, about 0.045 inches, about 0.05 inches, about 0.055 inches, about 0.06 inches, about 0.065 inches, about 0.07 inches, or ranges between any two of aforementioned values. The circumferential width of the bundles can each be greater than about 0.07 inches or less than about 0.04 inches. The circumferential width of each of the bundles 260, 270, 280, 290 can vary depending on the thickness of the wires 220, the thickness of the cords 230, or a jacket or shield thickness.

The bundles 260, 270, 280, 290 can each have a central axis. The first bundle 260 can have a first central axis, while the second bundle 270 a second central axis, the third bundle 280 a third central axis, and the fourth bundle 290 a fourth central axis. The central axes of the bundles 260, 270, 280, 290 can be different. For example, the first central axis of the first bundle 260 is different from the second, third, and fourth central axis of the bundles 270, 280, 290. The bundles 260, 270, 280, 290 can be placed within the cable 200 such that they are not concentric.

Some of the bundles of the cable 200 can be grouped together. For example, the first bundle 260 and the second bundle 270 can be grouped together and encased within an insulator or a separator. Similarly, the third bundle 280 and the fourth bundle 290 can be grouped together and encased with another insulator or another separator.

The bundles 260, 270, 280, 290 can carry different signals. For example, the wires 220 of the first bundle 260 can transmit signals between the detectors 126 of the noninvasive optical sensor 104 and the front end 116. The wires 220 of the second bundle 270 and the fourth bundle 290 can transmit signals between the emitter drivers 114 and the emitters 122 of the noninvasive optical sensor 104. The emitters 122 can be a light emitting diode (LED) that has a cathode side and an anode side, where a voltage difference between the cathode side and the anode side can facilitate generation of light. The wires 220 of the second bundle 270 can transmit signals between the cathode sides of the emitters 122 and the emitter driving circuit 114, while the wires 220 of the fourth bundle 290 can transmit signal between the anode sides of the emitters 122 and the emitter driving circuit 114. The wires 220 of the third bundle 280 can transmit signals between the memory device 132 of the noninvasive optical sensor 104 and the microprocessor 118 of the patient monitor 102.

The insulators 224 of the wires 220 can have different colors. The different colors can denote different signals the wires 220 carry. For example, one the insulators 224 that are green can denote one of the wires 220 that carries an input signal for the emitters 122, one of the insulators 224 that is white can denote one of the wires 220 that carries an output signal from the detector 126, one of the insulators 224 that is black may indicate that one of the wires 220 that does not carry any signal, and one of the insulators 224 that is purple or pink may indicate one of the wires 220 that carries a signal related to a thermometer. The wires 220 having the insulators 224 in different colors can further assist in a manufacturing process for the cable 200.

The bundles 260, 270, 280, 290 can allow one-way or two-way signal transmission between the noninvasive optical sensor 104 and the patient monitor 102. For example, a first of the wires 220 of the first bundle 260 can transmit signal to the detectors 126 while a second of the wires 220 of the first bundle 260 can receive signal from the detectors 126. In another example, the wires 220 of the second bundle 270 and the fourth bundle 290 can each communicate signals between the cathode/anode sides of the emitters 122 and the emitter driving circuit 114. The wires 220 of the third bundle 280 can each communicate signals between the memory device 132 and the microprocessor 118.

The cable 200 can include additional bundles for transmitting additional patient sensor data or parameters between the noninvasive optical sensor 104 and the patient monitor 102. For example, the noninvasive optical sensor 104 can include the temperature sensor 130, and the cable 200 can include a bundle with one or more of the wires 220 or the cords 230 to transmit signals between the temperature sensor 130 and the microprocessor 118.

The cords 230 can have a coextruded PVC sheath for reducing triboelectric noise generated by frictional contact between different cable elements. The cords 230 may not carry electronic signals. The cords 230 may drain triboelectric induced charges away from the insulator 224 as well as or better than the graphite coating and PVC sheath. As with the PVC sheath, grouping of the cords 230 with the wires 220 can increase the eventual signal quality output from signal processing circuitry, such as, for example, a differential amplifier. For example, use of the cords 230 in a manner that maintains the close physical proximity of the wires 220 tends to ensure external noise applied to the cable 200 is applied substantially equally (or common) to each of the wires 220. Thus, a differential amplifier (not shown) of the patient monitor 102 can effectively filter the applied external noise through, for example, the amplifier's common mode rejection.

Thus, while potentially exhibiting the same or superior advantageous characteristics of the coating or sheath, the cords 230 can be easier to control, cause less rigidity (for example, result in a more flexible bundle), and provide more straightforward processes during manufacturing than the coating or sheath. For example, the cords 230 may be simply cut away at points of connectivity for the wires 220 to circuit substrates or other electrical components. Moreover, the bundles 260, 270, 280, 290 can be made with the cords 230 that are hallow or thinner than the foregoing coatings or sheath. For these and other reasons, the cords 230 may provide for less expensive manufacturing processes.

The first bundle 260 can include an inner shield 226 and an inner jacket 228. The inner shield 226 and the inner jacket 228 can at least partially surround the wires 220 or the cords 230 of the first bundle 260. The inner shield 226 can advantageously reduce electromagnetic interference (EMI) between each of and crosstalk between the wires 220 or the cords 230. The inner shield 226 can be circumferentially surrounded by the inner jacket 228. One or more or all of the bundles 260, 270, 280, 290 can include the inner shield 226 and the inner jacket 228.

The inner shield 226 can be constructed of conductive materials or other suitable shield materials to meet performance or design objectives. Copper, silver, or other suitable materials can be used as materials for the inner shield 226. For example, the inner shield 226 can be constructed using braided copper strands. In another example, the inner shield 226 can be constructed using spiral copper strands. The thickness of the inner shield 226 can also be set to meet design objectives. The inner shield 226 can range in size from 44 AWG to 40 AWG. For example, the inner shield 226 is 44 AWG, tinned copper, with a ninety percent minimum coverage.

The inner jacket 228 can be designed to meet certain design or performance objectives. The inner jacket 228 can be constructed out of the jacket materials previously disclosed or other suitable materials. For example, the inner jacket 228 can be constructed from polytetrafluoroethylene, or PFTE, which allows the bundles 260, 270, 280, 290 to move more freely within the outer shield 206 with a decreased amount of friction between cable elements (for example, bundles 260, 270, 280, 290, the core 210, and the outer shield 206). In this regard, the inner jacket 228 can increase the flexibility of the cable 200 during twisting or kinking motions and prevent kinks from developing within the cable 200 after repeated use.

The inner jacket 228 can be constructed by layering materials. The inner jacket 228 can be constructed with a single sintered PFTE wrap plus a single unsintered PFTE wrap or be PVC with a single PFTE wrap. The inner jacket 228 can range in size from 0.001 inches to 0.1 inches. For example, the inner jacket 228 ranges in size from 0.002 inches to 0.008 inches. In one implementation, the inner jacket 228 is a sintered PFTE film that is approximately 0.0012 inches thick and a single layer of unsintered PFTE film that is approximately 0.004 inches thick.

The core 210 can be positioned between the bundles 260, 270, 280, 290 as shown in FIG. 2A-2C. The bundles 260, 270, 280, 290 can wrapped, twisted, or braided around the core 210 such that the core 210 defines a central axis of the cable 200. The number of the bundles surrounding or wrapping the core 210 can vary depending upon desired cable thickness, shape, size such as outer diameter, rigidity or flexibility, conductive performance, cost, and the like. The bundles 260, 270, 280, 290 can be weaved around the core 210. The core 210 and any one of the bundles 260, 270, 280, 290 may or may not share the same axis with another of the bundles 260, 270, 280, 290. The core 210 and any one of the bundles 260, 270, 280, 290 may or may not be concentric with respect to one another.

It can be advantageous to have the core 210 define a central axis of the cable 200. When the core 210 defines the central axis of the cable 200, more of the tensile stress on the cable 200 can be placed on the core 210 than the wires 220 or cords 230. Therefore, having the core 210 at a center of the cable 200 can reduce the tensile stress on individual components of the cable 200, including each of the bundles 260, 270, 280, 290, and increase the durability of the cable 200. Having the core 210 can evenly distribute stresses on the bundles 260, 270, 280, 290, the wires 220, and the cords 230 away from the central axis of the cable 200 during a kinking motion. The stress on the bundles 260, 270, 280, 290 can be torsional, shear, or tensional stress.

The cross-section of the core 210 can vary. For example, the core 210 can have a cross-section that is substantially rectangular, circular, elliptical, or another shape. The cross-sectional dimension or area of the core 210 can vary along the length of the core 210. The dimensions of the core 210 can be substantially the same as the circumferential width of the bundles within the cable 200. The width of the core 210 can be less than or greater than the circumferential width of one of the bundles 260, 270, 280, 290.

The dimensions of the core 210 can affect the interactions between the bundles 260, 270, 280, 290. For example, if the core 210 has a width less than the circumferential width of one or more of the bundles 260, 270, 280, 290, the core 210 may not prevent the bundles 260, 270, 280, 290 from contacting each other. If the core 210 has a width greater than the circumferential width of one or more the bundles 260, 270, 280, 290, the core 210 may reduce the amount of or prevent the contact between the bundles 260, 270, 280, 290. The reduction in or prevention of the contact between the bundles 260, 270, 280, 290 can reduce the amount of stress or friction generated between the bundles 260, 270, 280, 290.

In some examples, a ratio of the width of the core 210 to the circumferential width of one of the bundles 260, 270, 280, 290 may affect the interactions between the bundles 260, 270, 280, 290. The ratio of the width of the core 210 to the circumferential width of one of the bundles 260, 270, 280, 290 can be between about 0.3 and about 0.7, between about 0.35 and about 0.65, between about 0.4 and about 0.6, between about 0.45 and about 0.55, between about 0.48 and about 0.52, or about 0.3, about 0.35, about 0.4, about 0.45, about 0.5, about 0.55, about 0.6, about 0.65, about 0.7, or ranges between any two of aforementioned values. The ratio of the width of the core 210 to the circumferential width of one of the bundles 260, 270, 280, 290 can be greater than about 0.7 or less than about 0.3. The ratio of the width of the core 210 to the circumferential width of one of the bundles 260, 270, 280, 290 can vary depending on the size of one or more of the bundles 260, 270, 280, 290, the size of the core 210, the desired flexibility or rigidity of the cable 200, the number of bundles in the cable 200, or the like.

The core 210 can include arcuate surfaces 212 (which can be concave) that interact with outer surfaces of the inner jacket 228. The arcuate surfaces 212 of the core 210 can increase the durability of the inner jackets 228 of the bundles 260, 270, 280, 290 and reduce the overall dimension of the cable 200. The interaction between the core 210 and the inner jackets 228 of the bundles 260, 270, 280, 290 (for instance, pressure from the bundles 260, 270, 280, 290 on the core 210) can cause the arcuate surfaces 212 to form on an outer surface of the core 210. The arcuate surfaces 212 of the core 210 can be formed at least from interaction between the core 210 and the bundles surrounding the core 210. The arcuate surfaces 212 may each have cross-sectional shapes that correspond to outer surfaces of one of the bundles 260, 270, 280, 290. The shapes of the arcuate surfaces 212 may be rigid or may change responsive to the interaction between the core 210 and one or more of the bundles 260, 270, 280, 290. The arcuate surfaces 212 can, as discussed herein, reduce the amount of contact between the bundles 260, 270, 280, 290 and thereby reduce the amount of friction or stress on the bundles 260, 270, 280, 290. This can increase durability of the bundles 260, 270, 280, 290 and the cable 200.

The core 210 can be composed of two or more threads wrapped or weaved around a single thread. For example, the core 210 is composed of three or more threads wrapped or weaved around a single thread.

A cross-sectional area of the core 210 of the cable 200 can vary depending on the dimensions of the bundles 260, 270, 280, 290, number of bundles in the cable 200, diameter of the cable 200, desired rigidity or flexibility, desired durability, cost of manufacturing, conductive performance, or the like. The core 210 can have smaller or larger cross-sectional area than the bundles.

The cable 200 can include more than one core 210. For example, the cable 200 having the bundles 260, 270, 280, 290, as shown in FIG. 2B, can include the core 210 in the middle and four additional cores located in spaces formed between two of the four bundles 260, 270, 280, 290 and the outer shield 206. Those four additional cores can reduce the amount of friction between the bundles 260, 270, 280, 290 or between the bundles 260, 270, 280, 290 and the outer shield 206, thus increasing durability of the bundles 260, 270, 280, 290 and the outer shield 206. The additional cores can have cross-sectional areas or dimensions that vary from or match that of the core 210. For example, the cross-sectional areas of the additional cores may be larger than, smaller than, or the same as that of the core 210.

The core 210 can be made out of materials with high flexibility and tensile strength. For example, the core 210 is made out Kevlar fibers. Placing the core 210 with high flexibility and tensile strength can be advantageous in providing an overall cable construction that is both durable and flexible. The durability and flexibility resulting from having the core 210 in the middle wrapped by bundles 260, 270, 280, 290 can be advantageous in emergency medical situations, where medical assessments and interventions are often made in challenging conditions for electrical cables, such as cable 144 in FIG. 1.

Placing the separator 204 between the outer shield 206 and the outer jacket 202 can be advantageous because it can allow the entire construction (for example, the bundles 260, 270, 280, 290, the core 210, and the outer shield 206) to be more flexible or move freely inside the outer jacket 202 itself. In addition, this configuration can prevent extruded plastic from the outer jacket 202 from penetrating the braids of the outer shield 206. The separator 204 can be made out of materials with high flexibility, chemical resistance, thermal resistance, or electrical resistance. For example, the separator 204 can be made out of polytetrafluoroethylene (PTFE).

The outer shield 206, like the inner shield 226, can reduce EMI between the bundles 260, 270, 280, 290 and with other cables. The outer shield 206 can be composed of braided, tinned copper stranding or braided tinsel-wire stranding, where tinsel-wire is produced by wrapping server strands of thin metal foil around a flexible nylon or textile core. Because the thickness of the foil may be relatively thin, a bend radius imposed on the thin metal foil can be much greater than the thickness of the foil. In this regard, tinsel-wire can have a low probability of metal fatigue and, if used, can provide high tensile strength without impairing flexibility.

The outer jacket 202 can be made out of one or more materials having high flexibility, chemical resistance, high tensile strength, high cut resistance, or elongation properties. Such properties can provide greater protection from kinking and bending due to material properties. For example, the outer jacket 202 can be made out of thermoplastic polyurethane. The outer jacket 202 can have a thickness ranging between about 0.018″ and about 0.040″, between about 0.02″ and about 0.038″, between about 0.022″ and about 0.036″, between about 0.024″ and about 0.034″, between about 0.026″ and about 0.032″, between about 0.028″ and about 0.030″, or about 0.018″, about 0.020″, about 0.022″, about 0.024″, about 0.026″, about 0.028″, about 0.030″, about 0.032″, about 0.034″, about 0.035″, about 0.036″, about 0.038″, about 0.040″, or ranges between any two of aforementioned values. The thickness of the outer jacket 202 can be less than 0.018″ or greater than about 0.040″.

The cable 200 can have an outer diameter ranging between about 0.1″ and about 0.4″, between about 0.125″ and about 0.375″, between about 0.15″ and about 0.35″, between about 0.175″ and about 0.325″, between about 0.2″ and about 0.3″, between about 0.225″ and about 0.275″, or about 0.1″, about 0.125″, about 0.15″, about 0.175″, about 0.2″, about 0.225″, about 0.235″, about 0.25″, about 0.275″, about 0.3″, about 0.325″, about 0.35″, about 0.375″, about 0.4″, or ranges between any two of aforementioned values. The outer diameter of the cable 200 can be less than 0.125″ or greater than about 0.40″.

### Terminology

The following description is merely illustrative in nature and is in no way intended to limit the disclosure, its application, or uses. For purposes of clarity, the same reference numbers will be used in the drawings to identify similar elements. It should be understood that steps within a method may be executed in different order without altering the principles of the present disclosure.

Conditional language used herein, such as, among others, “can,” “might,” “may,” “for example,” and the like, unless specifically stated otherwise, or otherwise understood within the context as used, is generally intended to convey that certain embodiments include, while other embodiments do not include, certain features, elements or states. Thus, such conditional language is not generally intended to imply that features, elements or states are in any way required for one or more embodiments or that one or more embodiments necessarily include logic for deciding, with or without author input or prompting, whether these features, elements or states are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Further, the term “each,” as used herein, in addition to having its ordinary meaning, can mean any subset of a set of elements to which the term “each” is applied.

Terms such as “substantially,” “about,” “approximately” or the like as used in referring to a relationship between two objects is intended to reflect not only an exact relationship but also variances in that relationship that may be due to various factors such as the effects of environmental conditions, common error tolerances, manufacturing variances, or the like. It should further be understood that although some values or other relationships may be expressed herein without a modifier, these values or other relationships may also be exact or may include a degree of variation due to various factors such as the effects of environmental conditions, common error tolerances, or the like. For example, when referring to measurements, about a specified measurement can, in some contexts, refer to a measurement variation of around equal to or less than ±10%, ±5%, ±2%, or ±1% (such as a variation of ±10%, ±5%, ±2%, ±1%, ±0.8%, ±0.5%, or ±0.3%) from the specified measurement.

Although the low noise oximetry cable including cords is disclosed with reference to few various examples, the disclosure is not intended to be limited thereby. For example, the cords may not be hollow, may include a conductor or other conductive materials, may include only conductors of any suitably flexible material. Moreover, use of blank hollow cords may advantageously apply flexibility in a wide variety of applications, including cabling for virtually any medically monitored signals such as those invasively or noninvasively acquired signals relating to heart or brain activity or condition, spinal activity or condition, circulation parameters, tissue health, or the like. Moreover, the cabling may include only one or more portions of the communication link between sensor components and monitor electronics. The cable may also be an integral part of a reusable, disposable or combination sensor. Moreover, the addition of cords for shielding sensitive cabling may advantageously be applied generally to any and all cabling environments, and particularly in environments susceptible to triboelectric noise.

Additionally, other combinations, omissions, substitutions and modifications will be apparent to the skilled artisan in view of the disclosure herein. Accordingly, the present disclosure is not intended to be limited by the examples, but is to be defined by reference to the appended claims.

Additionally, all publications, patents, and patent applications mentioned in this specification are herein incorporated by reference to the same extent as if each individual publication, patent, or patent application was specifically and individually indicated to be incorporated by reference.

1.-20. (canceled)

21. A cable comprising:
a plurality of bundles, each of the plurality of bundles contacting at least two other of the plurality of bundles and comprising a jacket that at least partially surrounds a plurality of wires;
a core comprising a plurality of threads wrapped or weaved around another thread, the core having a plurality of concave surfaces formed from contact with the plurality of bundles; and
an outer jacket at least partially surrounding the plurality of bundles and the core.

22. The cable of claim 21, wherein the core defines a central axis of the cable.

23. The cable of claim 21, wherein a first bundle of the plurality of bundles comprises an inner shield that at least partially surrounds its plurality of wires, the inner shield being configured to provide electromagnetic interference protection to the plurality of wires of the first bundle.

24. The cable of claim 21, wherein each of the plurality of bundles contacts no more than two bundles of the plurality of bundles.

25. The cable of claim 21, wherein no bundle of the plurality of bundles is concentric with another bundle of the plurality of bundles.

26. The cable of claim 21, wherein no bundle of the plurality of bundles is concentric with the core.

27. The cable of claim 21, wherein the plurality of bundles are twisted or weaved around the core.

28. The cable of claim 21, wherein the core comprises Kevlar fibers.

29. The cable of claim 21, wherein the core is flexible and configured to reduce a tensile stress exerted on the plurality of bundles.

30. The cable of claim 21, wherein the plurality of bundles comprises four bundles.

31. The cable of claim 21, further comprising an outer shield that at least partially surrounds the plurality of bundles and the core, the outer shield being positioned proximate the plurality of bundles and the core relative to the outer jacket.

32. The cable of claim 31, further comprising a separator positioned between the outer jacket and the outer shield, the separator being configured to allow at least some movement of the outer jacket relative to the outer shield.

33. A physiological sensor system comprising:
a noninvasive sensor comprising a detector configured to detect light attenuated by a body tissue of a patient and output a detector signal indicative of the light detected after attenuation by the body tissue;
a patient monitor configured to receive the detector signal and determine one or more physiological parameters for the patient from the detector signal; and
the cable of claim 21.

34. A method of manufacturing a cable, the method comprising:
assembling a plurality of bundles, each of the plurality of bundles comprising a jacket that at least partially surrounds a plurality of wires; and
placing the plurality of bundles and a core within an outer jacket so that (i) each of the plurality of bundles contacts at least two other of the plurality of bundles and (ii) each of the plurality of bundles contacts the core and forms a concave surface on the core, the core comprising a plurality of threads wrapped or weaved around another thread.

35. The method of claim 34, wherein the core defines a central axis of the cable.

36. The method of claim 34, wherein said assembling the plurality of bundles comprises assembling a first bundle of the plurality of bundles to comprise an inner shield at least partially surrounding its plurality of wires, the inner shield being configured to provide electromagnetic interference protection to the plurality of wires of the first bundle.

37. The method of claim 34, wherein each of the plurality of bundles contacts no more than two bundles of the plurality of bundles.

38. The method of claim 34, further comprising twisting or weaving the plurality of bundles around the core.

39. The method of claim 34, wherein said placing comprises placing the plurality of bundles so that no two of the plurality of bundles are concentric with one another.

40. The method of claim 34, wherein said placing comprises placing the plurality of bundles so that none of the plurality of bundles are concentric with the core.
